(19) United States
(12) Patent Application Publication
(10) Pub. No.: US 2011/0263505 A1
(43) Pub. Date: Oct. 27, 2011

(54) Whey Protein Hydrolysate Containing Tryptophan Peptide Consisting of Alpha Lactalbumin and the Use Thereof

(75) Inventors: Thomas Henle, Dresden (DE); Andreas Deussen, Dresden (DE); Melanie Martin, Dresden (DE)

(73) Assignee: TECHNISCHE UNIVERSITÄT DRESDEN, Dresden (DE)

(21) Appl. No.: 13/002,049

(22) PCT Filed: Jul. 2, 2009
(86) PCT No.: PCT/EP09/58328

§ 371 Date: Apr. 1, 2011

(30) Foreign Application Priority Data
Jul. 2, 2008 (DE) 10 2008 032 828.6

(51) Int. Cl. A61K 38/57; C07K 5/097; C07K 5/09; C07K 5/113; C07K 5/103; C07K 5/117; C07K 7/06; C12P 21/06; A61P 9/12; C07K 5/078; C07K 5/062; C07K 5/065; C07K 5/068; C07K 1/14; C07K 5/072

(52) U.S. Cl. 514/16.2; 548/496; 530/331; 530/330; 435/68.1

(57) ABSTRACT

The invention relates to a whey protein hydrolysate, in particular a hydrolysate consisting of whey protein enriched with α-lactalbumin and α-lactalbumin, and the use thereof for producing pharmaceuticals, anti-hypertensive agents, food supplements, foodstuffs and animal feed, and to pharmaceuticals, anti-hypertensive agents, food supplements, foodstuffs and animal feed produced in this manner. The whey protein hydrolysate according to the invention, which has an ACE inhibiting and anti-hypertensive action, contains a physiologically active quantity of at least one peptide containing tryptophan, preferably at least one of the bio-active dipeptides Ile-Trp and Trp-Leu, and can be obtained by the extensive hydrolysis of whey protein isolates or of pure α-lactalbumin.

The invention concerns whey protein hydrolysates, in particular hydrolysates from whey protein that is enriched with α-lactalbumin and from α-lactalbumin, and their use for producing medicaments, blood pressure lowering agents, dietary supplements, food products, and animal feed, and the medicaments, blood pressure lowering agents, dietary supplements, food products and animal feed produced in this way.

Peripheral arterial hypertension (high blood pressure) is a widespread disease which in Western industrial nations, beginning at age 50, affects every second adult (systolic blood pressure above 140 mm Hg). High blood pressure leads to several other secondary diseases, such as degenerative changes of the blood vessels (arteriosclerosis) with arterial obstructive disease (coronary heart disease, cerebral insufficiency, peripheral arterial obstructive disease), heart hypertrophy, heart attack, cardiac insufficiency as well as stroke. The risk of such complications increases almost exponentially with increase of the systolic and diastolic blood pressure, wherein no threshold value exists. In comparison to systolic blood pressure of 110 mm Hg, the risk to die of a heart attack doubles at a pressure of 145 mm Hg and triples at 160 mm Hg. An integral component of modern high blood pressure therapy is inhibition of the so-called rennin-angiotensin-aldosteron system by inhibiting the activity of the angiotensin converting enzyme (ACE), a method whose effectiveness has been documented comprehensively, since the beginning of the 1980s in connection with the use of the so-called ACE Inhibitors. Based on estimation, in Germany today approximately 20% of the population or every second adult of age 55 plus is treated with medicaments against blood pressure that is too high. The costs paid by the health-care system in the Federal Republic of Germany amounted to approximately 1.5 billion Euros in the year 2005, and approximately one half thereof is spent on prescribing ACE inhibitors (Schwabe U. and Pfaffrath E. Arzneiverordnungs-Report 2006. Springer, Berlin, 2006). In view of this background novel preventive paths are necessary in order to achieve a further reduction of cardiovascular deaths as a result of high blood pressure. This could be achieved by introducing more effective ingredients into food consumed daily in order to thus affect beneficially the age-dependent development of arterial blood pressure.

High blood pressure has also become a pronounced problem in veterinary medicine in recent years causing direct treatment costs for animal owners. Estimates show that in Germany there are approximately 23 million pets, of which approximately 5.3 million are dogs and 7.5 million are cats (statistical yearbook for Germany, 2006). Because of the increasing life span of these pets, high blood pressure is also increasingly becoming a medical problem.

In addition to individual social importance of keeping pets, there is also a significant economic effect associated therewith. For example, sales of animal feed in Germany in the year 2008 amounted to approximately 2.6 billion Euros of which much more than 90% accounts for sales of dog food and cat food (source: Industrieverband Heimtierbedarf IVH e.V., “The German PET Market—Structure and Sales Data” http://www.ivh-online.de/fileadmin/user_upload/German_Pet_Market_{—}2008_A4.pdf). Studies indicate that approximately 61% of cats and up to 93% of dogs as a result of their “optimal” living conditions and the resulting long life expectancy develop high blood pressure during the course of their life (Acierno M. J., Labato M. A. (2005) Hypertension in renal disease Clin. Tech. Small Anim. Pract. 20:23-30). In this context, high blood pressure occurs often in connection with age-related kidney diseases or other metabolic disorders which may cause kidney failure, blindness and neurological complications (Brown S. A., Henik R. A. (1998) Diagnosis and treatment of systemic hypertension. Vet. Clin. North Am. Small Anim. Pract. 28:1481-94).

In the meantime, several peptides have become known that exist in the structure of food proteins, especially in milk proteins, and may inhibit ACE in vitro (Saito T. Antihypertensive peptides derived from bovine casein and whey proteins. Adv. Exp. Med. Biol. 2008, 606, 295-317). The best-known ones in this context are the tripeptides Val-Pro-Pro and Ile-Pro-Pro that are formed when milk is fermented with special microorganisms (Lactobacillus helveticus LBK-16H, Aspergillus oryzae) by proteolytic decomposition from casein (Nakamura Y. et al. Purification and characterization of angiotensin I-converting enzyme inhibitors from sour milk. J. Dairy Sci. 1995, 78, 777-783). These peptides that are also known as “casokinines” or “lactotripeptides” can either be formed endogenously in sour milk products or can be added, after prior enrichment, to certain foods. Appropriate functional milk products for lowering high blood pressure have become commercially available in the meantime in Japan (“Calpis”) and also in several European countries (“Evolus” in Finland, Portugal, Switzerland).

Clinical studies indicate that the consumption of sour milk products that contain Val-Pro-Pro and Ile-Pro-Pro may achieve a significant lowering of the blood pressure in people suffering from high blood pressure (Hata Y. et al. A placebo-controlled study of the effect of a sour milk on blood pressure in hypertensive subjects. Am. J. Clin. Nutr. 1996, 64, 767-771, Mizushima S. et al. Randomized controlled trial of sour milk on blood pressure in borderline hypertensive men. Am. J. Hypertens. 2004, 17, 701-706, Seppo L. et al. A fermented milk high in bioactive peptides has a blood pressure-lowering effect in hypertensive subjects. Am. J. Clin. Nutr. 2003, 77, 326-330, Mizuno S. et al. Antihypertensive effect of casein hydrolysate in a placebo-controlled study in subjects with high blood pressure and mild hypertension. Brit. J. Nutr. 2005, 94, 84-91), even though recent studies are contradictory (Lee Y. M. et al. Effect of a milk drink supplemented with whey peptides on blood pressure in patients with mild hypertension. Eur. J. Nutr. 2007, 46, 21-27, Engberink M. F. et al. Lactotripeptides show no effect on human blood pressure: results from a double-blind randomized controlled trial. Hypertension 2008, 51, 399-405).

For the aforementioned reasons it is moreover expedient to employ the principle of blood pressure lowering by ACE-inhibiting peptides also in feed for animals. A use would basically be conceivable for all products (enrichment of complex canned food and dry food, special “snacks” etc.). These foods would be freely purchasable and could be obtained by animal owners at significantly reduced cost in comparison to medicaments prescribed by a veterinarian.

Current applications for utilization of the aforementioned peptides are characterized by the following disadvantages. For example, they use relatively inefficient, i.e., weak, ACE inhibitors. Therefore, for a biological effect relatively high concentrations of peptides must be adjusted in the food in question (for example, fermented milk products) which for cost reasons as well as for sensory reasons is disadvantageous because the peptides produced by hydrolysis of food proteins have a bitter and unpleasant taste and this makes the use in foods difficult.

EP 1 087 668 B1 discloses a method for producing hydrolyzed whey protein products containing bioactive peptides that are free of bitter aromas. For this purpose, the whey protein-containing substrate is hydrolyzed enzymatically until a degree of hydrolysis of maximally 10% is reached. A degree of hydrolysis of 10% corresponds to an average cleavage of 10% of the peptide bonds in the hydrolyzed protein. Also, a series of bioactive peptides contained in the inventive products are disclosed that have a length of between 2 and 19 amino acids and have been released from primary sequences of protease-peptone, β-lactoglobulin, glycomacropeptide and β-casein.

WO 01/85984 discloses a method for producing a composition with ACE-inhibiting action. Here, isolated whey protein product is proteolytically digested. No specific peptides are identified as having ACE-inhibiting action.

WO 2006/025731 discloses enzymatic production of a protein hydrolysate with ACE-inhibiting action by digestion of a β-lactoglobulin-containing substrate in two steps. In the first step, the β-lactoglobulin-containing substrate is digested by means of a broadband endoprotease, preferably alcalase; in the second step, the digestion is carried out by means of a proline-specific endoprotease. The invention discloses inter alia also precipitation of other whey proteins such as α-lactalbumin when using whey protein as a substrate in order to increase the proportion of β-lactoglobulin in the starting material.

EP 1 226 267 B1 discloses a method for producing a product, containing peptides with anti-hypertension action, by fermentation of a casein-containing starting material with a lactic acid bacterium. Subsequently, the peptide-containing fermentation product is isolated by nanofiltration and subsequent recovery of the retentate.

Sato et al. disclose various peptides with ACE-inhibiting action, inter alia also Ile-Trp, that have been obtained by proteolytic digestion of brown algae with the protease S amano (Sato M. et al. Angiotensin I-converting enzyme inhibitory peptides derived from wakame (Undaria pinnatifida) and their antihypertensive effect in spontaneously hypertensive rats. J. Agric. Food Chem. 2002; 50(21) 6245-52).

JP 2006096747 discloses the production of bioactive peptides with ACE-inhibiting action, inter alia also Ile-Trp, from muscle or liver of salmon by protease digestion.

Kuba et al. disclose the ACE-inhibiting dipeptide Trp-Leu in traditional Japanese foods that are produced by fermentation of soy products (Kuba et al. Angiotensin I-Converting Enzyme Inhibitory Peptides Isolated from Tufuyo Fermented Soybean Food, Biosci. Biotechnol. Biochem. 2003, 67 (6), 1278-1283).

The patent WO 2004/047566 discloses food compositions for patients with liver damage as well as patients after operations, with infections or scalding that, in addition to oil rich in fatty acids as well as milk lecithin or soy lecithin as lipid component, contain milk protein hydrolysate and protein obtained from fermented milk products as a protein component. The advantageous effect of this food composition is said to be based on inhibiting inflammation-enhancing cytokines such as TNF-alpha, IL-6. Individual peptides that are responsible for these actions are not identified. The hydrolysis is thus realized purely empirically and phenomenologically, without indication of a chemical-analytical target parameter (for example, no degree of hydrolysis, no ranges of molecular weight, no individual peptides).

Patent WO 2007/004876 A2 discloses ACE-inhibiting peptides from whey proteins but focuses however in this context on higher-molecular peptides (oligopeptides with more than 2 to 14 amino acids). The dipeptides Ile-Trp and Trp-Leu are however not mentioned.

The publication of Mullaly, M. M. et al. (Biol. Chem. Hoppe-Seyler, Vol. 377, pp. 259-260, April 1996) concerns synthetic peptides with ACE-inhibiting action that correspond to certain sequence sections of alpha-lactalbumin and beta-lactoglobulin.

These sequences however do not encompass the dipeptides Ile-Trp and Trp-Leu.

The abstract of Philanto-Leppälä A. et al. (Journal of Dairy Research (2000) 67 (1), pp. 53-64) concerns in general the ACE-inhibiting action of whey hydrolysates.

The currently employed peptides are primarily acting in an ACE-inhibiting way; an action on other target systems (endothelin converting enzymes, matrix metalloproteases) and thus a vessel-protective broadband action is not disclosed. Moreover, many of the peptides disclosed up to now are oligopeptides that are comprised of 3 and more amino acids. This results in a distinct hydrolysis lability in the small intestine and thus a bad oral availability.

Today, α-lactalbumin as a component of whey is obtained in cheese production daily in a quantity of several tons. A comprehensive utilization is currently not provided for. Value-added utilization of such a food protein (α-lactalbumin) that up to now has found little use in the food industry for producing functional foods would therefore be very desirable.

Novel, and the object of this patent, is the use of special tryptophan-containing peptides (hydrolysates) derived from α-lactalbumin, wherein the peptides have an ACE-inhibiting action and can be utilized as ingredients for functional foods, dietary supplements, or pharmaceutical preparations.

The invention disclosed herein has the object to generate highly efficient peptides, i.e., peptides that have strong ACE-inhibiting action and thus have potentially a blood pressure-lowering action, by proteolytic decomposition of α-lactalbumin, a whey protein that up to now has been largely unutilized, and to use them as bioactive ingredients for functional foods.

The invention has furthermore the object to make available products that contain tryptophan-containing peptides that, in addition to lowering blood pressure as a result of ACE inhibition, also contribute additionally to vessel protection by inhibiting further relevant target systems (ECE, MMP). With the herein described invention a broad product palette of functionally effective foods is made available.

Surprisingly, these objects are solved by the whey protein hydrolysates according to the invention.

The invention comprises a whey protein hydrolysate with ACE-inhibiting and anti-hypertension action that contains a physiologically effective quantity of at least one of the bioactive tryptophan-containing peptides with a sequence selected from SEQ ID Nos. 2 to 32. The protein hydrolysate according to the invention is obtainable by extensive hydrolysis of isolated whey protein products or pure 60 -lactalbumin. The term extensive hydrolysis in the meaning of the invention is to be understood such that the average molecular weights of the peptides in the hydrolysate is less than 4 kDa, preferably less than 1,000 Da, especially preferred less than 500 Da. This corresponds to a decomposition of the proteins to dipeptides up to pentapeptides. The hydrolysis is preferably carried out enzymatically.

As a starting material for the hydrolysis, whey is suitable as well as commercial whey powder, isolated whey protein material, whey protein preparations or α-lactalbumin obtained from whey that have been further industrially processed by method steps such as microfiltration, isoelectric precipitation, chromatography etc. The preferred starting material for the hydrolysis is α-lactalbumin. The protein α-lactalbumin is contained in bovine milk whey in a proportion of approximately 20% of the total whey protein and represents thus, behind β-lactoglobulin, the second most frequent whey proteins in bovine milk whey. Advantageously, the whey protein hydrolysate according to the invention makes use (“added value”) of this food protein that up to now has been little used in the food industry for producing functional foods. By hydrolysis of this “waste product”, biologically highly effective peptides are obtained that are useable as ACE inhibitors for prophylactically and pharmacologically affecting the blood pressure.

Surprisingly, in the whey protein hydrolysate according to the invention peptides are released by hydrolysis that are bioactive and contain tryptophan and have a length of two to five, preferably two to three, especially preferred two amino acids of the primary sequence of α-lactalbumin. The bioactive tryptophan-containing peptides according to the invention contain the amino acid tryptophan at a terminal position, i.e., either as a C-terminal or as an N-terminal amino acid. Preferably, the tryptophan is positioned at the C-terminus.

The sequences of the bioactive peptides according to the invention result thus from the primary sequence of the α-lactalbumin and comprises the corresponding tryptophan residues (W26, W60, W104, and W118) and 1 to 4 amino acids that are positioned in the α-lactalbumin sequence either N-terminally or C-terminally relative to the respective tryptophan residue.

The primary sequence of bovine α-lactalbumin (UniProtKB/Swiss-Prot P00711 [LALBA_BOVIN]) corresponds to SEQ ID No. 1:

(SEQ ID No. 1)
1 11 21 31 41
EQLTKCEVFR ELKDLKGYGG VSLPEWVCTT FHTSGYDTQA IVQNNDSTEY
51 61 71 81 91
GLFQINNKIW CKDDQNPHSS NICNISCDKF LDDDLTDDIM CVKKILDKVG
101 111 121
INYWLAHKAL CSEKLDQWLC EKL

When using bovine milk whey for producing the hydrolysates according to the invention the sequences of the bioactive peptides according to the invention result from the primary sequence of the bovine α-lactalbumin and comprise 1 to 4 amino acids either N-terminally or C-terminally relative to one of the contained tryptophans (W26, W60, W104, and W118) and the corresponding tryptophan.

The bioactive tryptophan-containing peptides according to the invention comprise thus preferably the dipeptides with the SEQ ID Nos. 2-8, the tripeptides with the SEQ ID Nos. 9-16, the tetrapeptides with the SEQ ID Nos. 17-24, and the pentapeptides with the SEQ ID Nos. 25-32.

The sequences of the tryptophan-containing peptides according to the invention are listed in the following table.

| SEQ | Sequence | Sequence |
|---|---|---|
| ID-No. | (3-letter code) | (1-letter code) |
| 2 | Glu-Trp | EW |
| 3 | Trp-Val | WV |
| 4 | Ile-Trp | IW |
| 5 | Trp-Cys | WC |
| 6 | Tyr-Trp | YW |
| 7 | Trp-Leu | WL |
| 8 | Gln-Trp | QW |
| 9 | Pro-Glu-Trp | PEW |
| 10 | Trp-Val-Cys | WVC |
| 11 | Lys-Ile-Trp | KIW |
| 12 | Trp-Cys-Lys | WCK |
| 13 | Asn-Tyr-Trp | NYW |
| 14 | Trp-Leu-Ala | WLA |
| 15 | Asp-Gln-Trp | DQW |
| 16 | Trp-Leu-Cys | WLC |
| 17 | Leu-Pro-Glu-Trp | LPEW |
| 18 | Trp-Val-Cys-Thr | WVCT |
| 19 | Asn-Lys-IIe-Trp | NKIW |
| 20 | Trp-Cys-Lys-Asp | WCKD |
| 21 | Ile-Asn-Tyr-Trp | INYW |
| 22 | Trp-Leu-Ala-His | WLAH |
| 23 | Leu-Asp-Gln-Trp | LDQW |
| 24 | Trp-Leu-Cys-Glu | WLCE |
| 25 | Ser-Leu-Pro-Glu-Trp | SLPEW |
| 26 | Trp-Val-Cys-Thr-Thr | WVCTT |
| 27 | Asn-Asn-Lys-Ile-Trp | NNKIW |
| 28 | Trp-Cys-Lys-Asp-Asp | WCKDD |
| 29 | Gly-Ile-Asn-Tyr-Trp | GINYW |
| 30 | Trp-Leu-Ala-His-Lys | WLAHK |
| 31 | Lys-Leu-Asp-Gln-Trp | KLDQW |
| 32 | Trp-Leu-Cys-Glu-Lys | WLCEK |

It is especially preferred that the whey protein hydrolysates according to the invention contain the dipeptide Ile-Trp, which is up to now the most potent ACE inhibitor (IC₅₀=0.7 μM) detected in foods and/or the also highly effective dipeptide Trp-Leu (IC₅₀=10 μM). The dipeptides that are contained in the whey protein hydrolysate according to the invention and that, up to now, have not yet been known from α-lactalbumin are characterized by a strong ACE-inhibiting action and exhibit the highest effect described in connection with food peptides up to now.

The bioactive tryptophan-containing peptides have a structural relationship with ACE to inhibitors. As a result of this structural relationship, a comprehensive physiological effect, i.e., vessel protection, reduction of heart mass, regression of the vessel wall hypertrophy, protection against heart attack and stroke in addition to ACE-inhibiting action and interrelated blood pressure lowering rate can be derived. This effect was also suggested by tests on spontaneously hypertensive rats with a whey protein hydrolysate that contains these peptides.

The whey protein hydrolysate according to the invention is characterized by the IC₅₀ value of ACE-inhibiting action. The lower the IC₅₀ value, the higher the ACE-inhibiting action. The whey protein hydrolysate according to the invention has preferably an IC₅₀ value of 50 to 100 mg of whey protein hydrolysate/liter, preferably of 20 to 50 mg whey protein hydrolysate/liter, especially preferred of 2 to 20 mg whey protein hydrolysate/liter.

As a result of the low IC₅₀ value and the strong ACE-inhibiting action, very minimal quantities of peptide additives (for Ile-Trp only approximately 1/10, compared with the aforementioned tripeptides Ile-Pro-Pro and Val-Pro-Pro) are moreover required which, with regard to possible sensory aspects, is advantageous. Protein hydrolysates are usually imparted with a bitter taste as a result of hydrolysis which significantly limits their use when producing food products that are suitable for consumption or medications that are to be orally administered. Because of the bitter taste, protein hydrolysates can be added only in very limited quantities to these products. The whey protein hydrolysate according to the invention is however effective in already very small quantities and can therefore be used advantageously for producing food products and medications that are to be orally administered.

The highly effective peptides that are contained in the whey protein according to the invention are comprised preferably of highly hydrophobic amino acids, for example, the peptides with the SEQ ID Nos. 3, 4, 7, 11, 14, 17, and 22.

This makes the tryptophan-containing peptides advantageously stable with regard to hydrolysis so that even for extended length of time of hydrolysis it can be assumed that the hydrolysate contains effective concentrations of the tryptophan-containing peptides according to the invention.

The invention comprises moreover a method for producing the whey protein hydrolysates according to the invention. The hydrolysis is preferably enzymatically performed with at least the enzymes alcalase and trypsin. The digestion with the two enzymes can be carried out sequentially or simultaneously. The enzyme/substrate ratio (g enzymes/g substrate) is preferably between 1:10 and 1:10,000.

It is especially preferred that digestion with further enzymes will follow or further enzymes are added to the digestion with alcalase and trypsin. The employed enzymes are preferably endoproteases, especially preferred the enzymes are chymotrypsin, pancreatin, pepsin and CorolasePP.

The speed of hydrolysis depends on the employed enzymes as well as on their concentration and hydrolysis temperature. Also, further parameters may have an effect on the speed of hydrolysis, for example, the pH value or the chemical composition of the hydrolysis substrate.

The hydrolysis is carried out until more than 50% of the contained protein has a molecular weight of less than 4 kDa, preferably less than 1 kDa, and especially preferred less than 500 Da. This corresponds to a decomposition of the proteins to dipeptides up to pentapeptides. The dipeptides Ile-Trp and Trl-Leu according to the invention, for example, have a molecular weight of approximately 317 Da. By a sufficient length of time of carrying out the hydrolysis, it is ensured that a proportion as high as possible of the bioactive tryptophan-containing peptides is achieved in the whey protein hydrolysate according to the invention. The enzymatic hydrolysis is carried out at a temperature between 10° C. and 80° C., preferably between 30° C. and 70° C., especially preferred between 50° C. and 60° C. The progress of hydrolysis is monitored, for example, by gel permeation chromatography (GPC) and, after reaching the desired level of hydrolysis, is stopped by inactivating the enzymes, preferably by heating to a temperature of 80° C. to 110° C. The hydrolysis step (incubation time) lasts preferably 12 h to 36 h, more preferred 18 h to 30 h, especially preferred 24 h to 28 h.

The invention comprises also the use of whey protein for producing the whey protein hydrolysate according to the invention. The bioactive tryptophan-containing peptides according to the invention are contained in the primary sequence of the whey protein α-lactalbumin and are released from it by the enzymatic hydrolysis. Preferably, for the production of the whey protein hydrolysate α-lactalbumin-enriched whey protein, especially preferred α-lactalbumin itself as a whey protein, is used therefore for the production of the whey protein hydrolysate.

The invention comprises furthermore a method for producing bioactive tryptophan-containing peptides with a sequence selected from SEQ ID Nos. 2 to 32 with ACE-inhibiting and anti-hypertensive action. The method comprises in this connection the hydrolysis of whey protein and the subsequent isolation of the bioactive tryptophan-containing peptides from the whey protein hydrolysate. Preferably, the hydrolysis is carried out in this context according to the method disclosed in claims 7 to 10.

Suitable as a starting material for the hydrolysis is whey, commercially available whey powder, isolated whey protein material, whey protein preparations or α-lactalbumin obtained from whey that have been industrially further processed by microfiltration, isoelectric precipitation, chromatography etc. The invention concerns therefore also the use of whey protein for producing the bioactive tryptophan-containing peptides.

For producing the hydrolysates according to the invention in principle any type of mammalian milk is suitable as long as a satisfactory contents of α-lactalbumin (approximately 100 mg/liter) is contained in the whey. Preferably, however, bovine milk whey is employed.

The isolation of the bioactive tryptophan-containing peptide is realized by standard techniques of preparative chemistry, for example, extraction with organic solvents, ultrafiltration or preferably by preparative RP HPLC (reversed phase high-performance liquid chromatography).

The tryptophan-containing peptides isolated from whey protein hydrolysate can be added to food products. The food products include foodstuffs such as groceries and luxury foods as well as dietary supplements. Finally, the new effective tryptophan-containing peptides derived from α-lactalbumin are suitable for the pharmaceutical preparation of ACE inhibitors.

The invention comprises also the use of the whey protein hydrolysate according to the invention for producing food products. Moreover, the invention comprises the use of the whey protein hydrolysate according to the invention for producing a blood pressure-lowering medicament.

The invention comprises also the use of a bioactive tryptophan-containing peptide produced according to claim 11 for producing a food product.

Finally, the invention concerns the use of a bioactive tryptophan-containing peptide produced according to claim according to claim 11 for producing a blood pressure-lowering medicament. The medicament according to the invention is suitable especially for oral administration and can be prepared in any formulation suitable for oral administration. Suitable administration forms are, for example, powder, instant powder, pressed bodies, granules, tablets, effervescent tablets, capsules, coated tablets, syrup or the like.

The whey protein hydrolysates containing bioactive tryptophan-containing peptides described herein can be added directly to food products (for example, milk drinks and whey drinks, fruit juices, soft drinks).

The whey protein hydrolysates can also be dried by freeze-drying or spray-drying and processed to a powder. They are useable in this context as ingredients that are portioned and/or used in formulations with other dietary supplements such as vitamins, minerals, and/or trace elements. The powders are suitable as an additive for the food products and, moreover, for use as loose powders in order to generate granules, tablets, capsules, lozenges, sweets and liquids. Optionally, the powder can have additives and binders added to it.

The whey protein hydrolysate according to the invention is preferably offered as an instant product (soluble drink powder, for example, a drink powder for a cocoa, coffee or tea product, syrup, concentrate, effervescent powder or effervescent tablet) that is packaged in portioned packages of approximately 5 to 50 g, preferably 5 to 10 g, and, for example, is to be dissolved in 250 ml of water or fruit juice. The instant powder contains the whey protein hydrolysate according to the invention preferably in a quantity of 50% by weight up to 98% by weight, preferably of 70% by weight to 95% by weight, especially preferred 80% by weight to 92% by weight, relative to the total weight of the instant powder.

In this connection, the whey protein hydrolysate itself can serve as a carrier material for further components of the instant powder; the instant powder however may also contain further conventional carrier materials. In addition to the whey protein hydrolysate, the instant powder contains optionally minerals, trace elements, vitamins, natural and artificial sweeteners, aromas, acidifiers, carbonate compounds, coloring agents, preservatives, antioxidants, stabilizers and/or other food additives.

The manufacture of the instant drink powders is known to a person of skill in the art and is carried out in accordance with standard procedures.

Analog procedures can be employed for the substitution of the peptides in dry food/pellets for animals.

The invention concerns moreover a food product, containing the whey protein hydrolysate according to the invention or at, least one bioactive tryptophan-containing peptide produced according to the method according to the invention.

The food products are, for example, baked goods, in particular bread, cookies, pastry, long-keeping baked goods, crackers and waffles; desserts, in particular pudding, cream, and mousse; spreads for bread, margarine products, shortening; fruit products, in particular preserves, jams, jellies, canned fruit, fruit pulp, fruit juices, fruit juice concentrates, fruit-based soft drinks and fruit powder; vegetable products, in particular canned vegetables, vegetable juices and vegetable pulp; cereals, granola, and cereal mixtures; or sweets such as chocolate, hard candy, chewing gum, sugar-coated candy, licorice, marshmallow-type products, flakes, and nougat products.

These food products concern preferably a milk-based product or milk product. The milk product is preferably selected from the group comprised of milk, spreads for use on bread and produced from milk, milk drinks and whey drinks, yoghurt and yoghurt drinks, and other refreshment drinks made from milk, as well as ice cream, products or preparations based on cream cheese, cheese, butter, kefir, curd cheese, sour milk, butter milk, cream, condensed milk, milk powder, whey, lactose, milk protein, low-fat butter/cream, whey mixture or milk fat.

The food products contain optionally supplements, auxiliaries and/or sweeteners. Supplements or auxiliaries are preferably selected from the group comprised of aromas, for example, vanilla; coloring agents; flavoring agents; emulsifiers, for example, lecithin; thickening agents, for example, pectin, carob gum or guar gum; antioxidants; preservatives; triglycerides; and natural or synthetic vitamins, for example, vitamin A, vitamin B₁, vitamin B₂, vitamin B₆, vitamin B₁₂, vitamin B complex, vitamin C, vitamin D, vitamin E and/or vitamin K. Sweeteners are preferably selected from sucralose, sodium cyclamate, acesulfame K, aspartame, saccharine, acesulfame, cyclamate, thaumatine and neohesperidin.

The nutrient-based supply of anti-hypertensively active ingredients is suitable advantageously as a complement to pharmacological treatment and supplements the palette of supportive (“life style”) methods, for example, limiting salt intake, sports, yoga, and further diet-based measures. The ACE-inhibiting peptides that are derived from proteolytic decomposition of whey products therefore contribute in functional foods significantly to prophylaxis and supportive treatment of high blood pressure.

Finally, the new effective tryptophan-containing peptides from α-lactalbumin are suitable for the pharmaceutical preparation of ACE inhibitors and are therefore useable in pharmaceutical therapy of high blood pressure, including vessel wall remodeling, reduction of heart mass, improvement of blood flow reserve, and lowering the heart attack risk and the stroke risk, as a mono preparation or a combination preparation.

The invention comprises therefore also a blood pressure-lowering medicament, containing the whey protein hydrolysate according to the invention or at least one bioactive tryptophan-containing peptide produced according to the method according to the invention. The blood pressure-lowering medicament may contain further active ingredients, preferably further ACE inhibitors or other blood pressure-lowering active ingredients. The medicament may moreover contain solvents, solubilizing agents, antioxidants, resorption enhancers and other additives. The medicament can be present in liquid or solid form and is preferably administered orally.

The whey protein hydrolysates according to the invention or the bioactive tryptophan-containing peptides produced according to the method of the present invention are suitable moreover as an additive to animal feed, as a dietary feed supplement (snack) for animals or as a medicament for veterinary use. The whey protein hydrolysates or the bioactive tryptophan-containing peptides produced according to the method of the present invention can be added directly or in dried form to solid feed, high-energy feed, concentrated high-energy feed, dietary feed supplements, drinking water, salt licks or premixed preparations for such formulations. The whey protein hydrolysates can also be mixed by the animal owner into the animal feed or the drinking water as a powder. Optionally, the powder contains in addition to the whey protein hydrolysate additives and auxiliaries. In particular, further additives can be selected from the group of minerals, vitamins, trace elements, sugar, malt, molasses, cereals, bran, seeds (in particular from oil plants), proteins, amino acids, salts, oils, fats, fatty acids, fruits, fruit parts or fruit extracts or mixtures thereof.

In this way, the feed for pets and production or farm animals, for example, horses, cows, donkeys, sheep, goats, dogs, cats, pigs, hares, rabbits, guinea pigs, hamsters or birds can be enriched with the whey protein hydrolysates according to the invention; likewise, feed for zoo animals or exotic animals, for example, monkeys and apes, zebras, antelopes, giraffes, predatory cats, buffalos and rodents, and many more, without being limited to the listed animals, can be enriched.

Up to 50% by weight of the whey protein hydrolysate according to the invention can be admixed to a feed formulation (as a feed or as a dietary supplement). Usually, the whey protein hydrolysate is however admixed in a range of 0.01% up to 20% by weight, preferably 0.05% up to 10% by weight, especially preferred 0.05 to 5% by weight, into the compositions. The quantity of the whey protein hydrolysate to be admixed and its particle size depends on the type of animal in question.

When the whey protein hydrolysate, optionally together with at least one of the further above mentioned additives, is provided as a dietary feed supplement, this is either done immediately as a mixture of the components without preceding packaging or in the form of granules, pressed bodies, pellets, powders, coated tablets, syrup, a suspension or any other suitable administration form.

Preferably, the dietary feed supplement containing the whey protein hydrolysate according to the invention is provided in a form that enables the owner of the animal to administer an individual dose for each animal. This is, for example, especially provided for in case of a powder or granular material but also in case of coated tablets or pellets.

The invention encompasses also a method for treatment of high blood pressure, including vessel wall remodeling, reduction of heart mass, improvement of the blood flow reserve, and lowering the heart attack risk and stroke risk, characterized in that regularly a pharmaceutically effective dose of the whey protein hydrolysate according to the invention is administered.

With the aid of the attached illustrations the embodiments will be explained in more detail.

It is shown in:

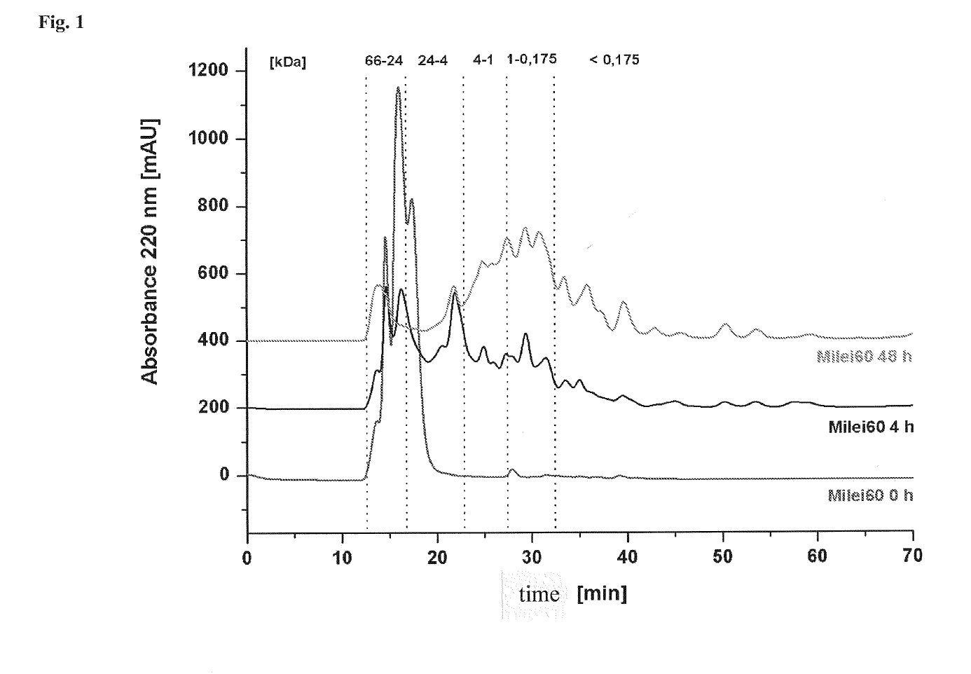

FIG. 1 GPC chromatographs of the molecular weight distribution of native (Milei60 0 h) as well as of hydrolyzed whey protein after 4 h (Milei60 4 h) and 48 h (Milei60 48 h of incubation time;

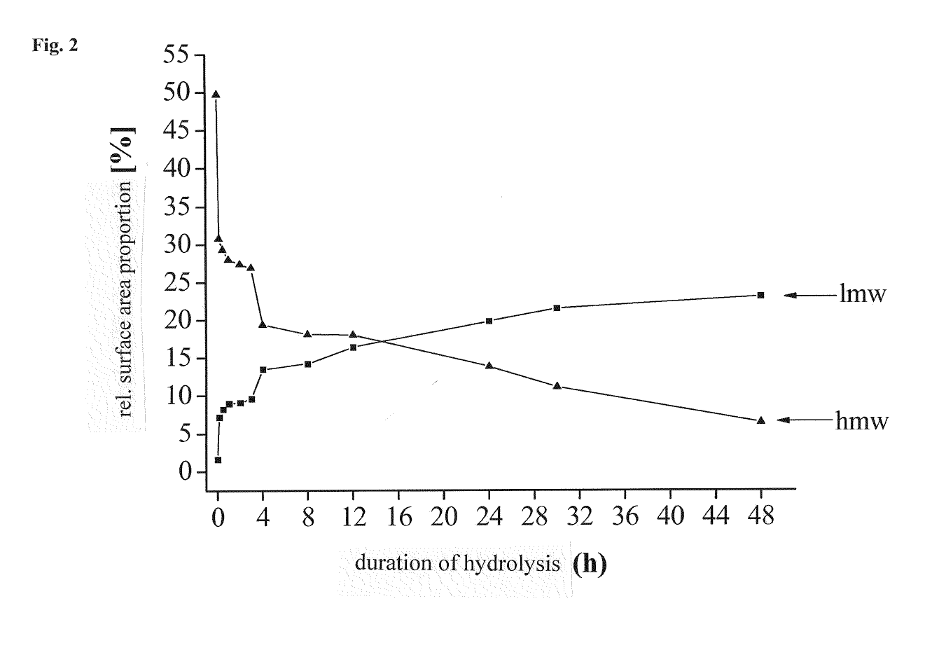

FIG. 2 shows the increase of low molecular weight (lmw) as well as decrease of high molecular weight (hmw) with increasing length of time of hydrolysis;

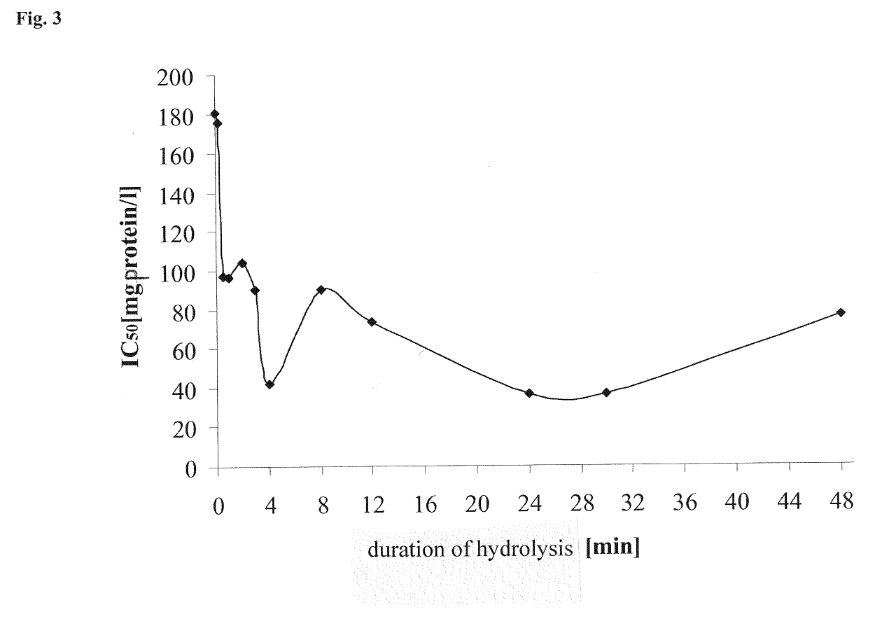

FIG. 3 shows the IC₅₀ value of ACE inhibiting action of the whey protein powder Milei60 as a function of the length of time of hydrolysis.

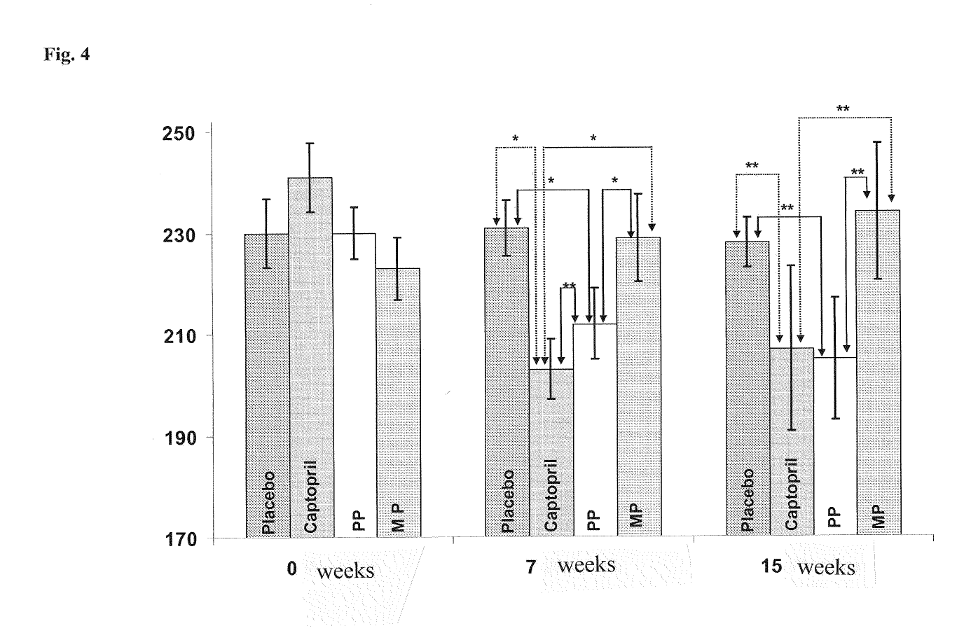

FIG. 4 shows the systolic blood pressure of spontaneously hypertensive rats at the beginning (0 weeks), after 7 and 15 weeks of a feed study. Administered was a preparation that contained the peptides according to the invention (PP), feed with intact whey protein (MP) as well as, as a control, a rat feed with captopril, a known ACE inhibiting agent as well as a placebo. PP leads to lowering of blood pressure that is comparable to that of captopril.

### EXAMPLE 1

### Enzymatic Hydrolysis of the Whey Protein Powder Milei60

The whey protein Milei60 was hydrolyzed enzymatically by proteolytically acting enzymes, i.e., trypsin obtained from porcine pancreas (EC 3.4.21.4) as well as two further proteases, i.e., Alcalase® (Bacillus licheniformis) and Flavourzyme (Aspergillus oryzae). The following Table illustrates how hydrolysis is carried out.

TABLE 1
Procedure of Enzymatic Hydrolysis of the Whey Protein Powder Milei60

| | Whey Protein Solution |
|---|---|
| | Milei60 10 g/50 ml |
| Method Steps | double distilled water |
| Adjusting pH with 3N NaOH | pH = 7.0 |
| addition of enzyme | Trypsin: 0.0015 g (2.338 U) |
| | Alcalase: 200 μl (0.07 U) |
| | Flavourzyme: 100 μl (7.5 U) |
| Length of time of incubation | 10 min, 30 min, 1 h, 2 h, 4 h, 8 h, 12 h, |
| | 30 h, 48 h |
| Hydrolysis temperature | 55° C. in water bath |
| cooldown | <10° C. within 10 min |
| Inactivation | Heating at 100° C. for 10 min in a drying |
| | cabinet |

The degree of hydrolysis was analyzed via molecular weight distribution by gel permeation chromatography (GPC) of samples that were incubated for different lengths of time. The determination of the molecular weights was realized subsequently by comparison with standards. Calibration was done in the range of 89.1 Da (alanine) to 25.0 kDa (chymotrypsinogen) (see also Table 2). For determining the exclusion limit Blue Dextran was used. Because of the differences in the absorption capability, various concentrations of the standard substances were used. In the following, the standard substances required for calibration as well as their molecular weights and the concentrations used for chromatography and the retention times are represented in table form.

TABLE 2
Standard Substances used for GPC Calibration with Corresponding
Molecular Weights [Da], Retention Times [min], and
Employed Concentrations [mg/ml]

| | Molecular Weight | Concentration | Retention Time |
|---|---|---|---|
| Standard | [Da] | [mg/ml] | [min] |
| Blue Dextran | 1,000,000 | 1.0 | 14.21 |
| Chymotrypsinogen | 25,000 | 0.6 | 16.20 |
| Ribonuclease A | 13,700 | 0.8 | 17.49 |
| Bovine Insulin | 5,733 | 5.0 | 20.08 |
| PTHIKWGD | 953.07 | 2.0 | 30.55 |
| LG | 188.23 | 2.0 | 32.24 |
| GG | 132.12 | 2.0 | 33.29 |
| L | 131.18 | 4.0 | 33.11 |
| A | 89.09 | 4.0 | 33.33 |

For determining the degree of hydrolysis the same protein concentration of 10 mg/ml was selected for all samples. The phosphate-buffered saline solution was used for elution. The detection of the eluted substances was done at 220 and 280 nm. The employed chemicals as well as parameters are listed in the following.

TABLE 3
GPC Parameters

| | |
|---|---|
| Column: | Superdex Peptide HR 10/30 |
| Injection volume: | 20 μl |
| Column | Room temperature |
| temperature: | |
| Detection: | UV detector: 220 und 280 nm |
| Eluting agent: | Phosphate-buffered saline solution: 10 mM |
| | phosphate + 300 mM sodium chloride (pH 7.4) |
| | Dissolve 1.47 g Na₂HPO₄•2H₂O (8.28 mM), |
| | 0.23 g KH₂PO₄ (1.72 mM), 17.53 g NaCl (300 mM) |
| | and 0.20 g KCl (2.7 mM) in 800 ml highest purity |
| | water, fill up with highest purity water to 1.0 l, |
| | membrane filtration (pH of the solution is |
| | automatically 7.4 and must thus not be adjusted) |
| Flow rate: | 0.5 ml/min |
| Elution system: | isocratic; 70 min |

The course of hydrolysis of the whey powder Milei60 was examined over 48 hours. The level of enzymatic digestion was determined by means of GPC and subsequent UV detection at 220 nm as well as 280 nm. For a characterization of the distribution of the molecular weights, the sample chromatographs were divided into five sections (66-24 kDa, 24-4 kDa, 4-1 kDA, 1-0.175 kDa, and <0.175 kDa); these areas are identified in FIG. 1. In this way, it is, for example, possible to indicate whether there is still intact protein present or whether during hydrolysis primarily amino acids, short-chain or long-chain peptides are released which, in turn, provides information in regard to the formation of possible ACE inhibiting agents.

FIG. 1 shows the distribution of the molecular weights of some selected hydrolysis stages (4 h, 48 h) as well as of the undigested whey protein powders (0 h). In the starting protein, as expected, several high molecular compounds (66-24 kDa) exist.

These are primarily the main proteins α-lactalbumin, present largely in its monomeric form, and β-lactoglobulin. The latter constitutes the primary component of proteins in Milei60 and exists primarily as a dimer.

However, other whey proteins, for example, the bovine serum albumin (BSA) which is eluted in the frontal area, but also like glycosylized forms of the whey protein components are also detected with this method. Smaller substances are not present or not present in relevant amounts in the product. Already after only ten minutes (see Table 4) a relatively strong decomposition of the whey proteins has taken place, in particular of α-lactalbumin; this decomposition advances continuously over the course of enzymatic digestion but even after 48 h complete hydrolysis has not yet been reached. During the course of time of hydrolysis, the proportion of compounds is shifted more and more toward the low molecular range where primarily dipeptides as well as free amino acids exist. For improved illustration, the distributions of molecular weights in the hydrolysates that result from the percentage of the surface areas of the GPC chromatographs are balanced in Table 4.

TABLE 4
Percentage of Surface Area Proportions of Corresponding
Molecular Weight Areas of Whey Protein Samples Hydrolyzed
for Different Lengths of Time.

| | Percentage of Surface Area Proportion of | | | | |
|---|---|---|---|---|---|
| | Corresponding Molecular Weight Ranges | | | | |
| Sample | 66-24 kDa | 24-4 kDa | 4-1 kDa | 1-0.175 kDa | <0.175 kDa |
| native | 47.9 | 39.6 | 1.0 | 1.4 | 1.6 |
| 10 min | 30.8 | 37.5 | 11.7 | 12.7 | 7.2 |
| 30 min | 29.3 | 36.9 | 12.1 | 13.5 | 8.2 |
| 1 h | 28.0 | 34.7 | 12.8 | 15.5 | 9.0 |
| 2 h | 27.4 | 34.4 | 13.6 | 15.5 | 9.1 |
| 3 h | 26.9 | 33.6 | 14.1 | 15.9 | 9.6 |
| 4 h | 19.4 | 29.6 | 17.4 | 20.1 | 13.5 |
| 8 h | 18.1 | 26.1 | 19.3 | 22.3 | 14.2 |
| 12 h | 18.0 | 23.0 | 19.3 | 23.3 | 16.4 |
| 24 h | 13.8 | 15.0 | 21.5 | 29.9 | 19.8 |
| 30 h | 11.1 | 13.4 | 22.1 | 31.9 | 21.5 |
| 48 h | 6.4 | 8.2 | 28.9 | 33.4 | 23.1 |

This also illustrates the continuous shift from the high molecular range to the low molecular range during continuing hydrolysis wherein a complete decomposition is not achieved. Finally, after 48 hours of enzymatic digestion the greatest proportion is not found in the range of <0.175 kDa in which primarily free amino acids exist but compounds with a molecular weight between 1 and 0.175 kDa constitute the highest proportion in the hydrolysate.

The decomposition of higher molecular compounds (24-66 kDa) or the release of the low molecular substances (<0.175 kDa) levels out significantly after a time of 24 hours of hydrolysis when compared to the preceding hours (see FIG. 2).

From a certain time on, no linear decomposition and thus release of smaller compounds occurred but after approximately 18 hours a plateau is observed, i.e., the hydrolysis occurred only relatively slowly. The strongest hydrolysis occurred within the first hours, and after four hours 51% of the peptides below 4 kDa were present.

### EXAMPLE 2

### Determination of ACE Inhibiting Effect of the Protein Hydrolysates

The determination of the ACE activity of the protein hydrolysates was carried out similar to a test disclosed by Cushman and Cheung in 1979 (Cushman, D. W. and Cheung, H.-S. Spectrophotometric Assay and Properties of Angiotensin-Converting Enzyme of Rabbit Lung, Biochemical Pharmacology, 1971, 20, 1637-1648). However, several modifications were carried out. The employed ACE is the rabbit lung enzyme obtained from Sigma-Aldrich. As a substrate N-benzoyl-glycyl-L-histidyl-L-leucin (HHL) was used which is considered to be analog to the natural substrate angiotensin I. ACE catalyzes the hydrolysis to L-histidyl-L-leucin (HL) and N-benzoyl-glycin (hippuric acid):

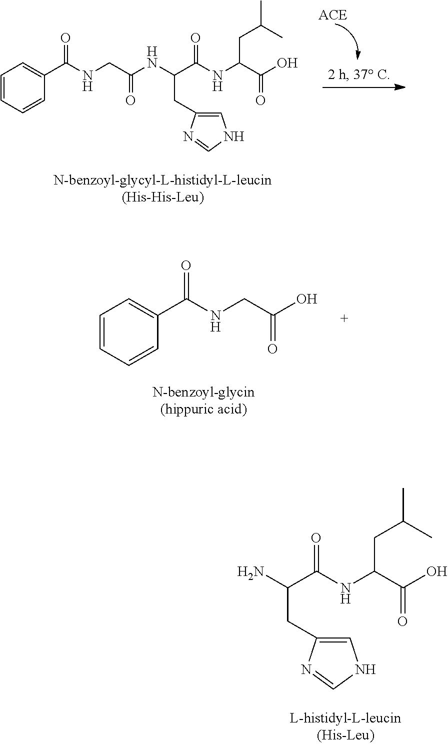

Since hippuric acid is UV-active (λₘₐₓ=228 nm), it is possible, based on its contents determined by RP HPLC, to define the conversion rate and thus the enzyme activity. By means of a concentration series of the respective inhibitor solution, the inhibitor concentration can be determined that is required for lowering the ACE activity by one half (IC₅₀).

In Table 5 the required reagents for performing the activity tests are described.

TABLE 5
Reagents for Determining the ACE Activity.

| | |
|---|---|
| Buffer: | 50 mM HEPES + 300 mM sodium chloride |
| | (pH 8.3 at 37° C.) |
| | Dissolve 2.383 g HEPES and 3.506 g NaCl in 190 ml |
| | highest purity water, heat to 37° C., adjust pH of the |
| | solution with NaOH to 8.3, fill up with highest purity water |
| | to 200 ml |
| Substrate: | 5 mM Hip-His-Leu in HEPES buffer |
| Enzyme: | ACE solution |
| | Dissolve 0.25 U in 4 ml double distilled water |
| Hydrochloric | 1N Dissolve 8.3 ml 37% HCl in highest purity water, |
| acid: | fill up to 100 ml with highest purity water |

All test solutions were dissolved in double distilled water.

Table 6 shows how the ACE activity test is performed. In each series of measurements two enzyme blind values were carried along in which, in place of the sample solution, the same volume of double distilled water had been added. The blind value corresponds to 100% ACE activity. Calculation of the IC₅₀ values was done by the software program SigmaPlot 5.0.

TABLE 6
Procedure for the ACE Activity Test

| | Blind value | Sample |
|---|---|---|
| Added substrate | 100 μl | |
| (HHL) | | |
| Addition of sample | 25 μl double distilled water | 25 μl sample solution |
| solution | | |

| | |
|---|---|
| Tempering of batch | 10 min at 37° C. |
| Enzyme addition | 20 μl ACE solution (1.25 mU) |
| Incubation | 2 h at 37° C. |
| Stopping the | 125 μl 1N HCl |
| reaction | |

In FIG. 7 the RP HPLC parameters for quantifying the hippuric acid as well as the parent solution required for calibration with corresponding dilution levels are listed.

TABLE 7
RP HPLC Parameters for Quantifying Hippuric Acid

| | |
|---|---|
| Parent solution | 1 mM hippuric acid solution: dissolve 17.92 mg |
| | hippuric acid in highest purity water |
| | and fill up subsequently to 100 ml. |
| | Calibration solutions: 0.005; 0.01; 0.025; 0.05; 0.1 mM |
| Column: | Supersphere 100, RP 18 endcapped; 125 × 4.6 mm, |
| | Packing diameter 4 μm |
| Injection | 20 μl |
| volume: | |
| Column | 25° C. |
| temperature: | |
| Flow rate: | 1.0 ml/min |
| Detection: | UV: 228 nm |
| Elution system: | Eluent A: 10 mM potassium dihydrogen phosphate |
| | solution (pH 3.0) |
| | Dissolve 1.3609 g KH₂PO₄ in 800 ml highest purity |
| | water, adjust pH of the solution with H₃PO₄ to 3.0, fill |
| | up with highest purity water to 1 l, membrane filtration |
| | Eluent B: 100% methanol (HPLC grade), degased |
| Gradient: | See Table 8 |

Table 8 shows the gradient that has been used for determining the hippuric acid contents by means of RP HPLC.

TABLE 8
RP HPLC Gradient Parameters for Determining
the Hippuric Acid Contents

| Time [min] | Eluent A [%] |
|---|---|
| 0 | 85 |
| 5 | 60 |
| 7 | 20 |
| 9 | 20 |
| 10 | 85 |
| 11 | 85 |

In addition to the samples that had been incubated for different lengths of time, also a blind value sample was analyzed that contained all employed enzymes and the whey protein powder in the same concentration as the hydrolysis samples. However, after addition of all substances, the enzymatic action was inactivated immediately by 10-minute incubation at 100° C. Also, possible effects by individual compounds on ACE were tested. For this purpose, the enzymes were inactivated and individually tested with respect to possible ACE inhibiting action. The same procedure was carried out with the whey protein powder. Whey protein powder that was not heat-treated was also tested with respect to its inhibiting action. For the determination of the IC₅₀ value of the hydrolysis samples several concentrations were used in the ACE activity test. A protein concentration of 1000 mg protein/I was used as the basis. This parent solution was diluted 1:2, 1:5, 1:10, 1:20, 1:50, 1:100 and all solutions were tested with respect to their ACE inhibiting effect. Evaluation was also done with SigmaPlot 5.0.

In order to examine the possible effect of the employed hydrolysis enzymes trypsin and Alcalase as well as of the whey protein powder on the activity of ACE, the enzymes individually, in the mixture in which they were used, and a solution of Milei60 without added enzyme were heat-treated in analogy to the hydrolysates at 100° C. for 10 minutes. This was done in order to denature the proteases because otherwise also a digestion of ACE and thus inhibition thereof would have to be considered. The tests showed that neither the inactivated enzymes, individually or as a mixture, nor the protein had an effect on ACE. On the other hand, a solution of the whey protein powder and the three enzymes, which solution was incubated immediately for 10 minutes at 100° C. for inactivation of the enzymes, demonstrated a relatively strong ACE inhibiting action. Apparently, a few minutes were sufficient in order to cause a well measurable hydrolysis of the whey proteins and to release in this connection ACE-inhibiting peptides. This confirms the strong enzymatic decomposition that is achieved by the employed proteases.

During the further course of the study each produced hydrolysate showed an ACE-inhibiting potential. For a better comparison of the data, the IC₅₀ values of each sample were determined and they are listed in Table 9.

TABLE 9
Representation of the IC₅₀ Values of the Whey Proteins
Hydrolyzed for Different Lengths of Time

| Length of Time of | |
|---|---|
| Hydrolysis [h] | IC₅₀ [mg protein/l] |
| 0 | 180 |
| 0.17 | 176 |
| 0.5 | 97 |
| 1 | 96 |
| 2 | 104 |
| 3 | 90 |
| 4 | 42 |
| 8 | 90 |
| 12 | 74 |
| 24 | 36 |
| 30 | 36 |
| 48 | 76 |

The inhibiting potential of the hydrolysates increased within the first four hours continuously until for the 4 h hydrolysate a very low IC₅₀ value of only 42 mg protein/I was determined. Subsequently, the inhibiting effect initially decreased again. After eight hours of hydrolysis more than twice as much sample was required in order to obtain the same effect as for the 4 h hydrolysate. Surprisingly, the inhibiting effect then increased again and reached after 24 and 30 hours the strongest inhibiting action which however was reduced again after 48 hours of hydrolysis duration. These results are illustrated also in FIG. 3 where the IC₅₀ value of the hydrolysates is plotted against the duration of hydrolysis.

The oscillating course of the inhibiting potential during the course of hydrolysis of the whey proteins can be explained such that formation as well as decomposition of ACE inhibiting compounds may happen. Since the proteolytic decomposition over time becomes slower and slower, it can be assumed then that also the change of the ACE inhibiting potential with increasing duration of hydrolysis will progress more and more slowly. Surprisingly, the 24-hour hydrolysate provokes the same strong reduction of ACE activity as the 4 h hydrolysate. It is to be assumed in this connection that this effect is based on different inhibitors because with progressing incubation time the whey protein components become more easily accessible for the enzymes so that potent inhibitors such as, for example, Ile-Trp (α-La 59-60) can be released only at a later point in time. Also, in the 24 h hydrolysate more compounds with a molecular weight between 1 and 0.175 kDa were present than in the 4 h hydrolysate.

### EXAMPLE 3

The release of dipeptides Trp-Leu and Ile-Trp according to the invention during the course of hydrolysis was analyzed by GPC and LC-ESI-TOF-MS (liquid chromatography/electrospray ionization time of flight mass spectrometry; liquid chromatography/electrospray ionization time of flight mass spectrometry).

In order to unequivocally identify the dipeptides according to the invention, first a separation of a hydrolysate by means of RP HPLC with subsequent detection by UV at 220 and 280 nm was carried out. The molecular weights of the individual peaks were determined with a high-resolution ESI-TOF mass spectrometer. The principle of electrospray ionization (ESI) is based on the sample solutions being sprayed to form a fine mist from which the solvent may completely evaporate. By charge transfer the remaining sample molecules receive one or several protons from the solvent. The ions are subsequently separated by time of flight analyzer (time of flight=TOF) according to their mass/charge ratio (m/z). From the determined molecular weights with the program Data Explorer™ the possible amino acid compositions can be finally determined.

For this analytical method selected hydrolysates in a concentration of 40 mg/ml were used and membrane-filtrated. The employed HPLC parameters are listed in Table 10.

TABLE 10
HPLC Parameters for LC-ESI-TOF-MS

| | |
|---|---|
| Column: | Eurospher-100 C 18; 250 × 4.6 mm, Packing |
| | diameter 5 μm |
| Injected volume: | 50 μl |
| Colum | 35° C. |
| temperature: | |
| Flow rate: | 0.7 ml/min |
| Detection: | UV (λ = 220 and 280 nm) and MS |

Elution system:

| | |
|---|---|
| Eluent A: | Acetonitrile/5 mM Ammonium acetate buffer |
| | (pH 5.5) 84 + 16 (v/v) |
| | Mixing 840 ml acetonitrile (HPLC grade) with 160 ml |
| | eluent B, membrane filtration and degasing |
| Eluent B: | 5 mM ammonium acetate buffer (pH 5.5) |
| | Dissolve 0.385 g CH₃COONH₄ in 800 ml highest |
| | purity water, adjust pH of solution with 3% acetic acid |
| | to 5.5, fill up with highest purity water to 1 l, |
| | membrane filtration |

The analyzed masses were compared with standard peptides Ile-Trp and Trp-Leu (Sachem Distribution Services GmbH, Weil am Rhein, Germany) that were carried along. The injected volume of the sample was 50 μl. The gradient system is illustrated in Table 11.

TABLE 11
Parameters of the Gradient Elution of LC-ECI-TOF-MS Tests

| Time [min] | Eluent A [%] |
|---|---|
| 0 | 5 |
| 10 | 5 |
| 14 | 8 |
| 23 | 45 |
| 29 | 69 |
| 44 | 80 |
| 48 | 80 |
| 50 | 5 |
| 57 | 5 |

In Table 12 the parameters measured by RP HPLC and LC-ESI-TOF-MS for the dipeptides according to the invention are listed. The Table contains also the IC₅₀ values that were obtained for the dipeptides. The determination of enzyme-inhibiting activity was carried out as disclosed in Example 2; the dipeptides Ile-Trp and Trp-Leu (Bachem Distribution Services GmbH, Well am Rhein, Germany) were employed for this purpose.

In GPC the identification of the dipeptides was carried out by comparison with peaks of the standard peptides that were carried along.

TABLE 12
Retention Times (RP HPLC), Peptide Masses and ACE-inhibiting
Activity of the Tested Dipeptides

| Retention | | | | |
|---|---|---|---|---|
| Times | Detected | Theoretical | | |
| (RP-HPLC) | Peptide Masses | Peptide Masses | | IC₅₀ |
| [min] | M + H⁺ [Da] | M + H⁺ [Da] | Sequence | [μM] |
| 26.2 | 318.2 | 318.17 | Ile-Trp | 0.7 ± 0.3 |
| 27.3 | 318.2 | 318.17 | Trp-Leu | 10 ± 1.7 |

The strongly ACE-inhibiting peptide Ile-Trp was detected unequivocally in the hydrolysates after a hydrolysis duration of three hours. Therefore, it appears to be accessible for the proteases only after a certain time has lapsed; this is true also for Trp-Leu. These two peptides however appear to be stable with respect to further digestion to the amino acids because the two peptides were found by GPC in all subsequent hydrolysates.

### EXAMPLE 4

### Feeding Tests on Spontaneously Hypertensive Rats

The basic importance of whey protein hydrolysates containing the tryptophan-containing peptides of the present invention for affecting the arterial blood pressure was objectified in animal tests on spontaneously hypertensive rats (FIG. 4). Here, the whey protein hydrolysate in comparison to a control diet caused a significant lowering of systolic blood pressure by 21±6 mm Hg after 7 weeks. Feeding captopril (ACE inhibitor of the first generation) lowered over the same time period the blood pressure by 28±7 mm Hg.

Further beneficial effects of the whey protein hydrolysates after feeding the preparation with the tryptophan-containing peptides according to the invention caused in comparison to controls an 8% reduced heart mass (captopril: 16% reduction) and the coronary flow reserve was increased by 75% in comparison to control. This implies comprehensive heart-protective and vessel-protective effects caused by the whey protein hydrolysate.

What is claimed is:

1. Whey protein hydrolysate with ACE-inhibiting and anti-hypertensive action, comprising a physiologically effective quantity of at least one bioactive tryptophan-containing peptide comprised of a partial sequence of α-lactalbumin comprising a tryptophan at the C-terminus or N-terminus and 1 to 4 amino acids C-terminally or N-terminally of the tryptophan, obtained by extensive hydrolysis.

2. Whey protein hydrolysate according to claim 1, characterized in that the bioactive tryptophan-containing peptide or peptides are selected from SEQ ID Nos. 2 to 32.

3. Whey protein hydrolysate according to claim 1, characterized in that more than 50% of the protein contained in the hydrolysate has a molecular weight of less than 4 kDa.

4. Use of whey protein for producing a whey protein hydrolysate according to claim 1.

5. Use of whey protein for producing bioactive tryptophan-containing peptides with ACE-inhibiting and anti-hypertensive action comprising a sequence selected from SEQ ID Nos. 2 to 32.

6. Method for producing a whey protein hydrolysate with ACE-inhibiting and anti-hypertensive action, characterized in that the hydrolysis is carried out enzymatically with at least the enzymes Alcalase and trypsin.

7. Method according to claim 6, characterized in that the hydrolysis is carried out until more than 50% of the obtained protein has a molecular weight of less than 4 kDa.

8. Method according to claim 6, characterized in that the whey protein used for hydrolysis is enriched with α-lactalbumin.

9. Method according to claim 6, characterized in that α-lactalbumin is used as a whey protein.

10. Method according to claim 6, wherein the enzymatic hydrolysis is carried out between 10° C. and 80° C.

11. Method for producing bioactive tryptophan-containing peptides according to claim 1, wherein whey protein is initially hydrolyzed and the bioactive dipeptides are isolated from the whey protein hydrolysate.

12. Use of a whey protein hydrolysate according to claim 1 and/or of a bioactive tryptophan-containing peptide isolated from the whey protein hydrolysate in a medicament, a food product, a veterinarian medicament or animal feed.

13. Food product comprising a whey protein hydrolysate according to claim 1, and/or a bioactive tryptophan-containing peptide isolated from the whey protein hydrolysate.

14. Medicament comprising a whey protein hydrolysate according to claim 1 and/or a bioactive tryptophan-containing peptide isolated from the whey protein hydrolysate.

15. Animal feed comprising a whey protein hydrolysate according to claim 1 and/or a bioactive tryptophan-containing peptide isolated from the whey protein hydrolysate.


SEQUENCE LISTING (32 sequences)

SEQ ID NO: 1 - protein, 123 aa, Bos taurus
EQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 2 - protein, 2 aa, Artificial Sequence
  Peptide obtained by hydrolysis of alpha-lactalbumin
EW

SEQ ID NO: 3 - protein, 2 aa, Artificial Sequence
  Peptide obtained by hydrolysis of alpha-lactalbumin
WV

SEQ ID NO: 4 - protein, 2 aa, Artificial Sequence
  Peptide obtained by hydrolysis of alpha-lactalbumin
IW

SEQ ID NO: 5 - protein, 2 aa, Artificial Sequence
  Peptide obtained by hydrolysis of alpha-lactalbumin
WC

SEQ ID NO: 6 - protein, 2 aa, Artificial Sequence
  Peptide obtained by hydrolysis of alpha-lactalbumin
YW

SEQ ID NO: 7 - protein, 2 aa, Artificial Sequence
  Peptide obtained by hydrolysis of alpha-lactalbumin
WL

SEQ ID NO: 8 - protein, 2 aa, Artificial Sequence
  Peptide obtained by hydrolysis of alpha-lactalbumin
QW

SEQ ID NO: 9 - protein, 3 aa, Artificial Sequence
  Peptide obtained by hydrolysis of alpha-lactalbumin
PEW

SEQ ID NO: 10 - protein, 3 aa, Artificial Sequence
  Peptide obtained by hydrolysis of alpha-lactalbumin
WVC

SEQ ID NO: 11 - protein, 3 aa, Artificial Sequence
  Peptide obtained by hydrolysis of alpha-lactalbumin
KIW

SEQ ID NO: 12 - protein, 3 aa, Artificial Sequence
  Peptide obtained by hydrolysis of alpha-lactalbumin
WCK

SEQ ID NO: 13 - protein, 3 aa, Artificial Sequence
  Peptide obtained by hydrolysis of alpha-lactalbumin
NYW

SEQ ID NO: 14 - protein, 3 aa, Artificial Sequence
  Peptide obtained by hydrolysis of alpha-lactalbumin
WLA

SEQ ID NO: 15 - protein, 3 aa, Artificial Sequence
  Peptide obtained by hydrolysis of alpha-lactalbumin
DQW

SEQ ID NO: 16 - protein, 3 aa, Artificial Sequence
  Peptide obtained by hydrolysis of alpha-lactalbumin
WLC

SEQ ID NO: 17 - protein, 4 aa, Artificial Sequence
  Peptide obtained by hydrolysis of alpha-lactalbumin
LPEW

SEQ ID NO: 18 - protein, 4 aa, Artificial Sequence
  Peptide obtained by hydrolysis of alpha-lactalbumin
WVCT

SEQ ID NO: 19 - protein, 4 aa, Artificial Sequence
  Peptide obtained by hydrolysis of alpha-lactalbumin
NKIW

SEQ ID NO: 20 - protein, 4 aa, Artificial Sequence
  Peptide obtained by hydrolysis of alpha-lactalbumin
WCKD

SEQ ID NO: 21 - protein, 4 aa, Artificial Sequence
  Peptide obtained by hydrolysis of alpha-lactalbumin
INYW

SEQ ID NO: 22 - protein, 4 aa, Artificial Sequence
  Peptide obtained by hydrolysis of alpha-lactalbumin
WLAH

SEQ ID NO: 23 - protein, 4 aa, Artificial Sequence
  Peptide obtained by hydrolysis of alpha-lactalbumin
LDQW

SEQ ID NO: 24 - protein, 4 aa, Artificial Sequence
  Peptide obtained by hydrolysis of alpha-lactalbumin
WLCE

SEQ ID NO: 25 - protein, 5 aa, Artificial Sequence
  Peptide obtained by hydrolysis of alpha-lactalbumin
SLPEW

SEQ ID NO: 26 - protein, 5 aa, Artificial Sequence
  Peptide obtained by hydrolysis of alpha-lactalbumin
WVCTT

SEQ ID NO: 27 - protein, 5 aa, Artificial Sequence
  Peptide obtained by hydrolysis of alpha-lactalbumin
NNKIW

SEQ ID NO: 28 - protein, 5 aa, Artificial Sequence
  Peptide obtained by hydrolysis of alpha-lactalbumin
WCKDD

SEQ ID NO: 29 - protein, 5 aa, Artificial Sequence
  Peptide obtained by hydrolysis of alpha-lactalbumin
GINYW

SEQ ID NO: 30 - protein, 5 aa, Artificial Sequence
  Peptide obtained by hydrolysis of alpha-lactalbumin
WLAHK

SEQ ID NO: 31 - protein, 5 aa, Artificial Sequence
  Peptide obtained by hydrolysis of alpha-lactalbumin
KLDQW

SEQ ID NO: 32 - protein, 5 aa, Artificial Sequence
  Peptide obtained by hydrolysis of alpha-lactalbumin
WLCEK